(19) United States
(12) Patent Application Publication
(10) Pub. No.: US 2006/0257339 A1
(43) Pub. Date: Nov. 16, 2006

(54) Use of conductive polymer nanofibers for treating keratinous surfaces

(75) Inventor: Murat Quadir, Scotch Plains, NJ (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 11/433,226

(22) Filed: May 11, 2006

Related U.S. Application Data
(60) Provisional application No. 60/681,058, filed on May 13, 2005.

(51) Int. Cl. A61K 8/73

(52) U.S. Cl. 424/61; 424/70.17

(57) ABSTRACT

Disclosed are compositions for imparting a visual and cosmetic effect onto a keratinous substrate, the composition containing: (a) a cosmetically acceptable medium; (b) at least one conductive polymer nanofiber present in an amount sufficient to impart a visual and cosmetic effect to a keratinous surface when irradiated with a flash of light; and (c) optionally, at least one cosmetic additive for imparting an additional cosmetic effect onto the keratinous surface.

### BACKGROUND OF THE INVENTION

The invention relates to the use of a composition comprising at least one conductive polymer nanofiber in a cosmetically acceptable medium which, when flash-welded, gives visual and cosmetic effects to keratinous surfaces. The invention also relates to a method for depositing a polymer film on keratinous surfaces by flash welding, the film being formed from at least one conductive polymer nanofiber.

Products for giving the hair sheen or luster effects are known. Such products contain molecules, or even polymers, in dissolved, emulsified or dispersed form, in a cosmetic solvent. However, these products still do not give the hair the desired sheen or luster.

In order to obtain a sheen or luster effect on the hair, it is known practice to use compositions that are rich in lubricant hydrophobic substances, such as organic oils or waxes or silicones. However, in this case also, the sheen or luster effect obtained lacks intensity and generally gives the hair an artificial appearance.

In addition, such compositions, once applied to the hair, have the drawback of giving the hair a greasy or sticky feel, which is unsatisfactory.

There is thus a need to find cosmetic compositions capable of giving keratinous surfaces, in general, and hair, in particular, an intense and natural sheen or luster without having the drawbacks mentioned above.

In addition, in order to obtain particularly attractive effects, combining a visual effect such as the provision of sheen or luster with a coloration effect such as a coloration boost and color protection, UV protection, additional texture effect, and strengthening effect may occasionally prove to be advantageous as well.

In cosmetics, in the treatment of keratinous surfaces such as hair and nails, the ability to produce varying visual and cosmetic effects from a film deposited on the surface can be very desirable. US20040103486, the entire content of which is hereby incorporated by reference, describes the use of at least one soluble conductive polymer in a cosmetic composition to give a visual effect to keratin fibers. Moreover, U.S. Pat. No. 6,749,843, the entire content of which is also hereby incorporated by reference, describes a method of reducing the flyaway effect of hair by using a cosmetic composition comprising at least one electrically conductive polymer. Neither reference, however, refers to the conductive polymer as being nano-sized or forming a film nor to the method of flash welding to deposit a nanofiber film onto a keratinous surface.

### SUMMARY OF THE INVENTION

The invention relates to a composition containing:
(a) a cosmetically acceptable medium;
(b) at least one conductive polymer nanofiber present in an amount sufficient to impart a visual and cosmetic effect to a keratinous substrate upon exposure to a flash of light; and
(c) optionally, at least one cosmetic additive for imparting an additional cosmetic effect onto the keratinous substrate.

In addition, the invention relates to a process for imparting a visual and cosmetic effect onto a keratinous substrate involving:
(a) providing a keratinous substrate;
(b) providing a cosmetic composition containing:
   (i) a cosmetically acceptable medium;
   (ii) at least one conductive polymer nanofiber; and
   (iii) optionally, at least one cosmetic additive for imparting an additional cosmetic effect onto the keratinous substrate;
(c) contacting the keratinous substrate with the cosmetic composition to form a treated keratinous substrate; and
(d) irradiating the treated keratinous substrate with a flash of light.

### DETAILED DESCRIPTION

Other than in the operating examples, or where otherwise indicated, all numbers expressing quantities of ingredients or reaction conditions are to be understood as being modified in all instances by the term “about”.

Now, it has been found, entirely surprisingly and unexpectedly, and this forms the basis of the present invention, that it is possible to uniformly impart onto a keratinous substrate, such as hair for example, a sheen or luster that is substantially more intense, more natural and more attractive than that given with the prior art means, through the use of conductive polymer nanofibers which are flash-welded onto the keratinous substrate.

The invention also relates to a process for treating a keratinous substrate, and more particularly human hair, with a composition comprising said conductive polymer nanofibers, and then depositing a continuous polymer film on the substrate by flash-welding.

For the purposes of the present invention, the term “visual effect” encompasses sheen, luster, color, metallic, goniochromatic, shimmering, fluorescent, thermochromic and electrochromic effects.

For the purposes of the present invention, the term “cosmetic effect” encompasses soft and pleasant feel, suppleness, smooth feel, UV protection, strength and desirable texture effects.

Moreover, and more particularly, when a lock of hair is illuminated at an angle α, it should be noted that the sheen or luster corresponds to the light intensity reflected at the angle α. The angle α conventionally used for measuring this specular reflection, in other words sheen/luster, is 20° C. This provision of sheen or luster may be measured by using a glossmeter, as described, for example, in ISO standard 2813-1994 of the AFNOR (August 1994, amended in February 1997). The visual and cosmetic effects may also be measured by other appropriate quantitative and qualitative measurements known by those skilled in the art.

Conductive Polymer Nanofibers

Conductive polymers are well known to those skilled in the art and described especially in the book “Handbook of Organic Conductive Molecules and Polymers”—Wiley 1997—New York, Vol. 1, 2, 3, but also in the revue Can. J. Chem. Vol. 64, 1986, the entire contents of each of which is hereby incorporated by reference. Besides polymerization via chemical or electrochemical oxidation, they may also be obtained by polycondensation (dihalogenated thiophene; catalysis with nickel or palladium complexes); by Suzuki coupling (coupling between a halogen function, for example bromine, and a boronic acid, catalysis: complex of palladium and base; this then gives coupling of AA-BB type (reaction of monomers of the type A-X-A with B—X′—B) or of A-B type (reaction of several monomers of A-X—B type)); by Stille coupling (formation of a carbon-carbon bond in the presence of Pd-based catalysis of AA-BB or A-B type); by Reike polymerization (organozinc in the presence of a nickel complex); by McCulloch type polymerization, etc. The conductive polymers present in the composition according to the invention are moreover described in international patent application WO 99/47570, the entire content of which is hereby incorporated by reference.

In more recent years, nanostructures (nanofibers, nanowires, nanorods) of these conductive polymers have been developed which have more effective surface areas compared to the conventional conductive polymers. There are different synthetic approaches to preparing polyaniline nanostructures and in particular, polyaniline nanofibers (Huang et al., 2003, J. Am. Chem. Soc., 125, 314-315), the entire content of which is hereby incorporated by reference. It is such polyaniline nanofibers that have been observed to form a film of a random network of nanofibers on a substrate and furthermore, to form a smooth and continuous film upon flash-welding or ‘melting’ the nanofibers on the substrate.

An enhanced photothermal reaction has been observed in which the heat generated from a camera flash resulted in instantaneous welding or ‘melting’ of a random network of conducting polymer nanofibers, specifically, polyaniline nanofibers, to form a smooth and continuous film (Huang and Kaner, 2004, Nature Materials, 3, 783-786), the entire content of which is hereby incorporated by reference. The advantages of flash welding in being able to change the properties of polyaniline such as conductivity, surface area, optical absorption, permeability and thermal stability, and thus creating patterns on the film that result in certain reflectivity properties, are mentioned as well. Another observation is that although the change in the surface roughness or porosity of the flash-welded film results in a more hydrophobic surface, the film has been found to be easily washed off by water from the substrate. Conductive polyaniline nanofibers mixed with polystyrene microspheres were also shown to form a film by flash welding. It is thus believed that the nanofibers can be flash-welded with other cosmetic additives to form polymer-polymer and polymer-inorganic composites. The flash-welded blend of the conductive polyaniline nanofibers with a cosmetic additive chosen from a polymer or inorganic material can produce different film properties as in the polyaniline-polystyrene film that appears white due to strong light scattering.

For purposes of the present invention, the term “conductive polymer nanofibers” means a conductive polymer having a diameter of up to 100 nm, a length of up to 10 μm, a conductivity of 0.5 siemens/cm or lower and which is capable of forming a film, i.e., being crosslinked, when irradiated with a sudden flash of light.

For purposes of the present invention, the term “flash of light” means having a light power of 0.05 J/cm² and higher, a light wavelength of from 400 nm to 7 μm, a flash duration of 1.0 ms and greater, and a flash distance to the target of 1.0 cm and larger.

Examples of suitable conductive polymers from which nanofibers may be obtained include, but are not limited to, homopolymers and copolymers comprising polyaniline, polythiophene, polypyrrole, polyarylene, polyphenylene, poly(bisthiophenephenylene, conjugated ladder polymer, poly(arylene vinylene), poly(arylene ethynylene), polyfurans, paraphenylene sulfides, polyindoles, aromatic polyamides, aromatic polyazomethines, aromatic polyhydrazides, organometallic derivatives thereof.

Particularly preferred conductive polymer nanofibers are those derived from polyaniline.

The conductive polymer nanofibers are generally present in the composition in an amount of from 0.01% to 50% by weight, preferably from 0.01% to 2% by weight, and more preferably from 0.1% to 1% by weight, based on the weight of the composition.

Medium

The cosmetically acceptable medium of the cosmetic composition is preferably an aqueous medium consisting of water and may advantageously comprise at least one cosmetically acceptable organic solvent such as, for example, alcohols such as ethyl alcohol, isopropyl alcohol, benzyl alcohol and phenylethyl alcohol, or glycols or glycol ethers such as, for example, ethylene glycol monomethyl, monoethyl or monobutyl ether, propylene glycol or ethers thereof such as, for example, propylene glycol monomethyl ether, butylene glycol, dipropylene glycol and also diethylene glycol alkyl ethers, for instance diethylene glycol monoethyl ether or monobutyl ether, or alternatively polyols, for instance glycerol. Polyethylene glycols, polypropylene glycols and mixtures of all these compounds may also be used as the solvent.

The solvents may then be present in the composition in an amount of from 0.5% to 60% by weight, preferably from 1% to 20% by weight, and more preferably from 2% to 10% by weight, based on the weight of the composition.

Cosmetic Additives

The cosmetic composition of the present invention may also contain at least one cosmetic additive for imparting an additional cosmetic effect onto the keratinous substrate, wherein the cosmetic additive may be a polymer or an inorganic material.

Polymers

Suitable polymers which may be used as cosmetic additives include, but are not limited to, fixative polymers, conditioning polymers, water-soluble styling polymers, polymer beads, thickening agents, polyorganosiloxanes, and mixtures thereof.

Fixative Polymers

Use may be made in particular of fixative polymers selected from anionic, cationic, amphoteric and non-ionic polymers and mixtures thereof. The fixative polymer may additionally be halogenated, in particular fluorinated.

The fixative polymers can be used in solubilized form or else in the form of dispersions of solid polymer particles (latex or pseudo-latex).

The cationic fixative polymers which can be used according to the present invention can be selected from polymers containing primary, secondary, tertiary and/or quaternary amine groups forming part of the polymer chain or linked directly to it and having a molecular weight of between 500 and approximately 5,000,000 and preferably between 1000 and 3,000,000.

The anionic fixative polymers that are generally used are polymers containing groups derived from carboxylic, sulphonic or phosphoric acid and have a weight-average molecular weight of between approximately 500 and 5,000,000.

The carboxylic groups are provided by unsaturated mono- or dicarboxylic acids monomers such as those corresponding to the formula (II): 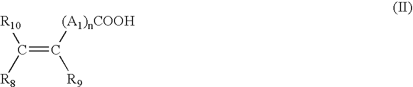
in which:
n is an integer from 0 to 10,
A₁ denotes a methylene group, optionally connected to the carbon atom of the unsaturated group, or to the neighboring methylene group when n is greater than 1, via a hetero atom such as oxygen or sulphur,
R₁₀ denotes a hydrogen atom or a phenyl or benzyl group,
R₈ denotes a hydrogen atom or a lower alkyl or carboxyl group, and
R₉ denotes a hydrogen atom, a lower alkyl group or a —CH₂—COOH, phenyl or benzyl group.

In the abovementioned formula, a lower alkyl radical preferably denotes a group having 1 to 4 carbon atoms and in particular methyl and ethyl.

The anionic fixative polymers containing carboxylic groups which can be used according to the invention are:
a) Homo- or copolymers of acrylic or methacrylic acid or salts thereof and in particular the products sold under the names VERSICOL E or K by the company Allied Colloid and ULTRAHOLD by the company BASF. The copolymers of acrylic acid and of acrylamide sold in the form of their sodium salt under the names RETEN 421, 423 or 425 by the company Hercules, the sodium salts of polyhydroxycarboxylic acids.
b) Copolymers of acrylic or methacrylic acids with a monoethylenic monomer such as ethylene, styrene, vinyl esters, acrylic acid esters or methacrylic acid esters. These copolymers can be grafted onto a polyalkylene glycol such as polyethylene glycol and optionally crosslinked. Such polymers are described in particular in French patent 1,222,944 and German patent application 2,330,956, the entire contents of which are hereby incorporated by reference. Mention may be made in particular of the copolymers containing an optionally N-alkylated and/or hydroxyalkylated acrylamide unit in their chain as described in particular in Luxembourg patent applications 75370 and 75371, the disclosure of which is specifically incorporated by reference herein, or sold under the name QUADRAMER by the company American Cyanamid. Mention may also be made of copolymers of acrylic acid and of C₁-C₄ alkyl methacrylate and terpolymers of vinylpyrrolidone, of (meth)acrylic acid and of (meth)acrylate of C₁-C₂₀ alkyl, for example lauryl (such as the product sold by the company ISP under the name ACRYLIDONE LM), tert-butyl (LUVIFLEX VBM 70 sold by BASF) or methyl (STEPANHOLD EXTRA sold by Stepan) and methacrylic acid/ethyl acrylate/tert-butyl acrylate terpolymers such as the product sold under the name LUVIMER 100 P by the company BASF.
c) Copolymers derived from crotonic acid such as those containing vinyl acetate or propionate units in their chain and optionally other monomers such as allylic esters or methallylic esters, vinyl ether or vinyl ester of a linear or branched saturated carboxylic acid with a long hydrocarbon chain such as those containing at least 5 carbon atoms, it being possible for these polymers optionally to be grafted and crosslinked, or alternatively a vinyl, allylic or methallylic ester of an α- or β-cyclic carboxylic acid. Such polymers are described, inter alia, in French patents 1,222,944, 1,580,545, 2,265,782, 2,265,781, 1,564,110 and 2,439,798, the disclosures of which are specifically incorporated by reference herein. Commercial products falling into this class are the resins 28-29-30, 26-13-14 and 28-13-10 sold by the company National Starch.
d) Copolymers derived from C₄-C₈ monounsaturated carboxylic acids or anhydrides selected from:
   copolymers comprising
   (i) one or more maleic, fumaric or itaconic acids or anhydrides and
   (ii) at least one monomer selected from vinyl esters, vinyl ethers, vinyl halides, phenylvinyl derivatives, acrylic acid and its esters, the anhydride functions of these copolymers optionally being monoesterified or monoamidated. Such polymers are described in particular in U.S. Pat. Nos. 2,047,398, 2,723,248 and 2,102, 113 and GB patent 839,805, the disclosures of which are specifically incorporated by reference herein, and in particular those sold under the names GANTREZ AN or ES and AVANTAGE CP and Aquaflex FX-64 by the company ISP;
   copolymers comprising
   (i) one or more maleic, citraconic or itaconic anhydrides and
   (ii) one or more monomers selected from allylic or methallylic esters optionally containing one or more acrylamide, methacrylamide, α-olefin, acrylic or methacrylic ester, acrylic or methacrylic acid or vinylpyrrolidone groups in their chain, the anhydride functions of these copolymers optionally being monoesterified or monoamidated.
These polymers are described, for example, in French patents 2,350,384 and 2,357,241, the entire contents of which are hereby incorporated by reference.
E) Polyacrylamides containing carboxylate groups.

The polymers comprising sulphonic groups are polymers containing vinylsulphonic, styrenesulphonic, naphthalenesulphonic or acrylamidoalkylsulphonic units.

These polymers can be selected in particular from: polyvinylsulphonic acid salts having a weight-average molecular weight ranging from approximately 1000 to approximately 100,000, as well as the copolymers with an unsaturated comonomer such as acrylic or methacrylic acids and their esters, as well as acrylamide or its derivatives, vinyl ethers and vinylpyrrolidone; polystyrenesulphonic acid salts, the sodium salts having a weight-average molecular weight of about 500,000 and about 100,000, which are sold respectively under the names FLEXAN 500 and FLEXAN 130 by National Starch. These compounds are described in patent FR 2,198,719, the disclosure of which is specifically incorporated by reference herein; polyacrylamidesulphonic acid salts, such as those mentioned in U.S. Pat. No. 4,128,631, the entire content of which is hereby incorporated by reference, and more particularly polyacrylamidoethylpropanesulphonic acid sold under the name COSMEDIA POLYMER HSP 1180 by Henkel.

Anionic fixative polymers may be also be sulphonated polyesters comprising repeating units representable by the following general formula: 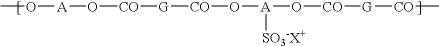
in which A and G represent divalent radicals and X represents an alkali metal, especially sodium or potassium. Among the preferred sulphonated polyesters according to the invention, A represents an arylene radical, especially phenylene, and G represents a linear or branched alkylene radical optionally interrupted by one or more oxygen atoms, or a cycloalkylene radical. When G represents a linear or branched alkylene radical optionally interrupted by one or more oxygen atoms, the radical —O-G-O— is preferably a (poly)alkylene glycol residue containing 1 to 20 alkylene glycol units. The alkylene radical is preferably, according to the invention, a lower, linear or branched C₂-C₄ alkylene radical, more preferably an ethylene radical. Such polymers are described in particular in the patents U.S. Pat. No. 3,546,008, U.S. Pat. No. 4,340,519, U.S. Pat. No. 3,734,874, U.S. Pat. No. 3,779,993, U.S. Pat. No. 4,233,196 and U.S. Pat. No. 5,386,003 and in the patent application WO 95/32997, the entire contents of which are hereby incorporated by reference. Among these polymers, preference will be given to those marketed under the names AQ 1045, AQ 1350 and AQ 14000 by the company EASTMAN CHEMICAL, more particularly AQ 1350.

According to the invention the anionic fixative polymers are selected from acrylic acid copolymers such as the acrylic acid/ethyl acrylate/N-tert-butylacrylamide terpolymers sold in particular under the name ULTRAHOLD STRONG by the company BASF, copolymers derived from crotonic acid such as the vinyl acetate/vinyl tert-butylbenzoate/crotonic acid terpolymers and the crotonic acid/vinyl acetate/vinyl neododecanoate terpolymers sold in particular under the name RESIN 28-29-30 by the company National Starch, polymers derived from maleic, fumaric or itaconic acids or anhydrides with vinyl esters, vinyl ethers, vinyl halides, phenylvinyl derivatives or acrylic acid and its esters, such as the methyl vinyl ether/maleic anhydride monoesterified copolymers sold, for example, under the name GANTREZ by the company ISP, the copolymers of methacrylic acid and of methyl methacrylate sold under the name EUDRAGIT L by the company Rohm Pharma, the copolymers of methacrylic acid/methyl methacrylate/C₁-C₄ alkyl acrylate/acrylic acid or C₁-C₄ hydroxyalkyl methacrylate which are sold in the form of dispersions under the name AMERHOLD DR 25 by the company Amerchol or under the name ACUDYNE 255 by the company Rohm & Haas, the copolymers of methacrylic acid and of ethyl acrylate sold under the name LUVIMER MAEX or MAE by the company BASF and the vinyl acetate/crotonic acid copolymers and the vinyl acetate/crotonic acid copolymers grafted with polyethylene glycol, sold under the name ARISTOFLEX A by the company BASF.

The anionic fixative polymers which are more preferred are selected from the monoesterified methyl vinyl ether/maleic anhydride copolymers sold under the name GANTREZ ES 425 by the company ISP, the acrylic acid/ethyl acrylate/N-tert-butylacrylamide terpolymers sold under the name ULTRAHOLD STRONG by the company BASF, the copolymers of methacrylic acid and of methyl methacrylate sold under the name EUDRAGIT L by the company Rohm Pharma, the vinyl acetate/vinyl tert-butylbenzoate/crotonic acid terpolymers and the crotonic acid/vinyl acetate/vinyl neododecanoate terpolymers sold under the name RESIN 28-29-30 by the company National Starch, the copolymers of methacrylic acid and of ethyl acrylate sold under the name LUVIMER MAEX or MAE by the company BASF and the vinyl pyrrolidone/acrylic acid/lauryl methacrylate terpolymers sold under the name ACRYLIDONE LM by the company ISP.

The amphoteric fixative polymers which can be used in accordance with the invention can be selected from polymers containing units B and C distributed randomly in the polymer chain, in which B denotes a unit derived from a monomer containing at least one basic nitrogen atom and C denotes a unit derived from an acid monomer containing one or more carboxylic or sulphonic groups, or alternatively B and C can denote groups derived from carboxybetaine or sulphobetaine zwitterionic monomers; B and C can also denote a cationic polymer chain containing primary, secondary, tertiary or quaternary amine groups, in which at least one of the amine groups bears a carboxylic or sulphonic group connected via a hydrocarbon radical or alternatively B and C form part of a chain of a polymer containing an α,β-dicarboxylic ethylene unit in which one of the carboxylic groups has been made to react with a polyamine containing one or more primary or secondary amine groups.

The amphoteric fixative polymers corresponding to the definition given above which are more particularly preferred are selected from the following polymers:
(1) Polymers resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylate and acrylate, dialkylaminoalkylmethacrylamides and -acrylamides. Such compounds are described in U.S. Pat. No. 3,836,537, the disclosure of which is specifically incorporated by reference herein.
(2) Polymers containing units derived from:
   a) at least one monomer selected from acrylamides and methacrylamides substituted on the nitrogen with an alkyl radical,
   b) at least one acidic comonomer containing one or more reactive carboxylic groups, and
   c) at least one basic comonomer such as esters containing primary, secondary, tertiary and quaternary amine substituents of acrylic and methacrylic acids and the product of quaternization of dimethylaminoethyl methacrylate with dimethyl or diethyl sulphate.

The N-substituted acrylamides or methacrylamides which are more particularly preferred according to the invention are groups in which the alkyl radicals contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are selected more particularly from acrylic acid, methacrylic acid, crotonic acid, itaconic acid, maleic acid and fumaric acid and alkyl monoesters, having from 1 to 4 carbon atoms, of maleic or fumaric acids or anhydrides.

The preferred basic comonomers are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer such as the products sold under the name AMPHOMER or LOVOCRYL 47 by the company National Starch are particularly used.
(3) crosslinked and alkylated polyamino amides partially or totally derived from polyamino amides of formula (III): [symbol]
   CO—R₁₀—CO-Z [symbol] (III)
in which:
R₁₀ represents a divalent radical derived from a saturated dicarboxylic acid, a mono- or dicarboxylic aliphatic acid containing an ethylenic double bond, an ester of a lower alkanol, having from 1 to 6 carbon atoms, of these acids or a radical derived from the addition of any one of the acids to a bis(primary) or bis(secondary)amine, and
Z denotes a bis(primary), mono- or bis(secondary) polyalkylene-polyamine radical and preferably represents:
a) in proportions of from 60 to 100 mol %, the radical
   —NH [symbol] (CH₃)ₓ—NH [symbol] ₚ (IV)
where x=2 and p=2 or 3, or alternatively x=3 and p=2 this radical being derived from diethylenetriamine, from triethylenetetraamine or from dipropylenetriamine;
b) in proportions of from 0 to 40 mol %, the radical (IV) above in which x=2 and p=1 and which is derived from ethylenediamine, or the radical derived from piperazine: 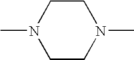
c) in proportions of from 0 to 20 mol %, the —NH—(CH2)6-NH— radical derived from hexamethylenediamine, these polyamino amines being crosslinked by addition of a difunctional crosslinking agent selected from epihalohydrines, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

The saturated carboxylic acids are preferably selected from acids having from 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the alkylation are preferably propane sultone or butane sultone, the salts of the alkylating agents are preferably the sodium or potassium salts.
(4) polymers containing zwitterionic units of formula (V): 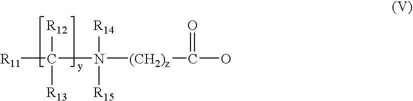
in which:
R₁₁ denotes a polymerizable unsaturated group such as an acrylate, methacrylate, acrylamide or methacrylamide group,
y and z represent an integer ranging from 1 to 3,
R₁₂ and R₁₁₃ represent a hydrogen atom, methyl, ethyl or propyl,
R₁₄ and R₁₅ represent a hydrogen atom or an alkyl radical such that the sum of the carbon atoms in R₁₄ and R₁₅ does not exceed 10.

The polymers comprising such units can also contain units derived from non-zwitterionic monomers such as dimethyl or diethylaminoethyl acrylate or methacrylate or alkyl acrylates or methacrylates, acrylamides or methacrylamides or vinyl acetate.

By way of example, mention may be made of the copolymer of methyl methacrylate/dimethyl carboxymethylammonio methyl ethylmethacrylate such as the product sold under the name DIAFORMER Z301 by the company Sandoz.
(5) Polymers derived from chitosan containing monomer units corresponding to the following formulae: 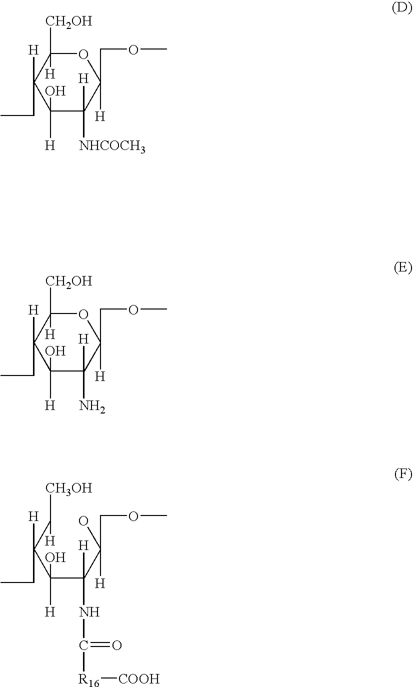
   the unit D being present in a concentration ranging from 0 to 30%,
   the unit E in a concentration ranging from 5 to 50%, and
   the unit F in a concentration ranging from 30 to 90%,
   it being understood that, in this unit F, R₁₆ represents a radical of formula: 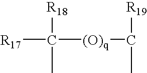
   in which,
   if q=0, R₁₇, R₁₈ and R₁₉ each independently represent a hydrogen atom, a methyl, hydroxyl, acetoxy or amino residue, a monoalkylamine residue or a dialkylamine residue which are optionally interrupted by one or more nitrogen atoms and/or optionally substituted with one or more amine, hydroxyl, carboxyl, alkylthio or sulphonic groups, an alkylthio residue in which the alkyl group bears an amino residue, at least one of the radicals R₁₇, R₁₈ and R₁₉ being, in this case, a hydrogen atom;
   or, if q=1, R₁₇, R₁₈ and R₁₉ each independently represent a hydrogen atom, as well as the salts formed by these compounds with bases or acids.
(6) Polymers derived from the N-carboxyalkylation of chitosan, such as N-carboxymethylchitosan or N-carboxybutylchitosan sold under the name “EVALSAN” by the company Jan Dekker.
(7) Polymers corresponding to the formula (VI) are described, for example, in French patent 1,400,366, the disclosure of which is specifically incorporated by reference herein: 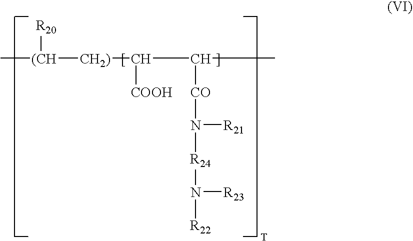
   in which:
   R₂₀ represents a hydrogen atom, a CH₃O, CH₃CH₂O or phenyl radical,
   R₂₁ denotes hydrogen or a lower alkyl radical such as methyl or ethyl,
   R₂₂ denotes hydrogen or a lower alkyl radical such as methyl or ethyl,
   R₂₃ denotes a lower alkyl radical such as methyl or ethyl or a radical corresponding to the formula: —R₂₄—N(R₂₂)₂, wherein R₂₂ can be the same or different,
   R₂₄ representing a —CH₂—CH₂—, —CH₂—CH₂—CH₂— or —CH₂—CH(CH₃)— group, and the higher homologues of these radicals containing up to 6 carbon atoms.
(8) Amphoteric polymers of the type -D-X-D-X selected from:
   a) polymers obtained by the action of chloroacetic acid or sodium chloroacetate on compounds containing at least one unit of formula:
      =D=X=D=X=D= (VII)
   where D denotes a radical 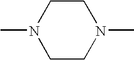
   and X denotes the symbol E or E′, E or E′, which may be identical or different, denotes a divalent radical which is an alkylene radical containing a straight or branched chain containing up to 7 carbon atoms in the main chain, which is unsubstituted or substituted with hydroxyl groups and which can contain, in addition to the oxygen, nitrogen and sulphur atoms, from 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups.
   b) Polymers of formula:
      -D-X-D-X— (VII)
   in which D denotes a radical 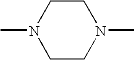
   and X denotes the symbol E or E′ and at least once E′, E having the meaning given above and E′ is a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily containing one or more carboxyl functions or one or more hydroxyl functions and betainized by reaction with chloroacetic acid or sodium chloroacetate.
(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also contain other vinyl comonomers such as vinylcaprolactam.

The amphoteric fixative polymers which are particularly preferred according to the invention are those of family (3), such as the copolymers whose CTFA name is octylacrylamide/acrylates/butylaminoethyl methacrylate copolymer, such as the products sold under the names AMPHOMER, AMPHOMER LV 71 or LOVOCRYL 47 by the company National Starch and those of family (4) such as the copolymer of methyl methacrylate/dimethyl carboxymethylammonio methyl ethylmethacrylate, sold, for example, under the name DIAFORMER Z301 by the company Sandoz.

The anionic or amphoteric fixative polymers can, if necessary, be partially or totally neutralized. The neutralizing agents are, for example, sodium hydroxide, potassium hydroxide, 2-amino-2-methyl-1-propanol, monoethanolamine, triethanolamine or triisopropanolamine and inorganic or organic acids such as hydrochloric acid or citric acid.

The non-ionic fixative polymers useful according to the present invention are, in particular, polyurethanes.

Conditioning Polymers

The conditioning polymers which can be used in accordance with the invention may be cationic or amphoteric conditioning polymers.

For the purposes of the present invention, the term “cationic conditioning polymers” means any polymer which comprises cationic groups or groups that can be ionized into cationic groups and which can improve the cosmetic properties of keratin fibres, in particular the disentangling, the softness, the sheen and the volume.

The cationic or amphoteric conditioning polymers that are suitable are advantageously chosen from those already known per se as improving the cosmetic properties of the hair, that is to say, especially, those described in patents and patent applications EP 337 354, FR 2 270 846, FR 2 383 660, FR 2 598 611, FR 2 470 596, FR 2 519 863, FR 2 788 974 and FR 2 788 976, the entire contents of which are hereby incorporated by reference.

However, more specific examples of cationic conditioning polymers that may especially be mentioned include cationic polymers comprising at least primary, secondary, tertiary and/or quaternary amine groups, which either may form part of the main polymer chain, or may be borne by a side substituent directly attached thereto.

Thus, mention may be made of:
(1) copolymers of acrylamide and of dimethylaminoethyl methacrylate quaternized with dimethyl sulphate or with a dimethyl halide (Hercofloc from Hercules); copolymers of acrylamide and of methacryloyloxyethyltrimethylammonium chloride (Bina Quat P 100 from Ciba Geigy); the copolymer of acrylamide and of methacryloyloxyethyltrimethylammonium methosulphate (Reten from Hercules); quaternized or non-quaternized vinylpyrrolidone/dialkylaminoalkyl acrylate or methacrylate copolymers (Gafquat range from ISP; Copolymer 845, 958 and 937 from ISP); dimethylaminoethyl methacrylate/vinylcaprolactam/vinylpyrrolidone terpolymers (Gaffix VC 713 from ISP); vinylpyrrolidone/methacrylamidopropyldimethylamine copolymers (Styleze CC 10 from ISP); vinylpyrrolidone/dimethylaminopropylmethacrylamide quaternized copolymers (Gafquat HS 100 from ISP);
(2) Cellulose ether derivatives comprising quaternary ammonium groups, as described in FR 1 492 597, the entire content of which is hereby incorporated by reference. These polymers are also defined in the CTFA dictionary as quaternary ammoniums of hydroxyethylcellulose that has reacted with an epoxide substituted with a trimethylammonium group;
(3) Cationic cellulose derivatives such as copolymers of cellulose or cellulose derivatives grafted with a water-soluble quaternary ammonium monomer, described especially in U.S. Pat. No. 4,131,576, the entire content of which is hereby incorporated by reference such as hydroxyalkylcelluloses, for instance hydroxymethylcellulose, hydroxyethylcellulose or hydroxypropylcellulose grafted especially with a methacryloylethyltrimethylammonium, methacrylamidopropyltrimethylammonium or dimethyldiallylammonium salt;
(4) The cationic polysaccharides described more particularly in patents U.S. Pat. No. 3,589,578 and U.S. Pat. No. 4,031,307, the entire contents of which are hereby incorporated by reference, such as guar gums containing trialkylammonium cationic groups. Guar gums modified with a salt, for instance the chloride, especially 2,3-epoxy-propyltrimethylammonium chloride, are used for example;
(5) Polymers consisting of piperazinyl units and of divalent alkylene or hydroxyalkylene radicals containing straight or branched chains, optionally interrupted with oxygen, sulphur or nitrogen atoms or with aromatic or heterocyclic groups, and also the oxidation and/or quaternization products of these polymers. Such polymers are described especially in FR 2 162 025 and FR 2 280 361, the entire contents of which are hereby incorporated by reference;
(6) Water-soluble polyaminoamides prepared in particular by polycondensation of an acidic compound with a polyamine, which are optionally crosslinked, optionally alkylated, or, if they comprise one or more tertiary amine functions, quaternized. These polymers are described especially in FR 2 252 840 and FR 2 368 508, the entire contents of which are hereby incorporated by reference;
(7) Polyaminoamide derivatives resulting from the condensation of polyalkylene polyamines with polycarboxylic acids followed by an alkylation with difunctional agents. Examples that may be mentioned include adipic acid-dialkylaminohydroxyalkyldialkylene-triamine polymers in which the alkyl radical is C₁-C₄. Such polymers are described especially in FR 1 583 363, the entire content of which is hereby incorporated by reference;
(8) Polymers obtained by reacting a polyalkylene polyamine comprising two primary amine groups and at least one secondary amine group with a dicarboxylic acid chosen from diglycolic acid and saturated C₃-C₈ aliphatic dicarboxylic acids, and then with epichlorohydrin. Such polymers are described especially in U.S. Pat. No. 3,227,615 and U.S. Pat. No. 2,961,347, the entire contents of which are hereby incorporated by reference;
(9) Cyclopolymers of alkyldiallylamine or of dialkyldiallylammonium, in homopolymer or copolymer form, as described in FR 2 080 759 and in its Certificate of Addition No. 2 190 406, the entire contents of which are hereby incorporated by reference;
(10) Diquaternary ammonium polymers as described in FR 2 320 330, FR 2 270 846, FR 2 316 271, FR 2 336 434, FR 2 413 907, U.S. Pat. No. 2,273,780, U.S. Pat. No. 2,375,853, U.S. Pat. No. 2,388,614, U.S. Pat. No. 2,454,547, U.S. Pat. No. 3,206,462, U.S. Pat. No. 2,261,002, U.S. Pat. No. 2,271,378, U.S. Pat. No. 3,874,870, U.S. Pat. No. 4,001,432, U.S. Pat. No. 3,929,990, U.S. Pat. No. 3,966,904, U.S. Pat. No. 4,005,193, U.S. Pat. No. 4,025,617, U.S. Pat. No. 4,025,627, U.S. Pat. No. 4,025,653, U.S. Pat. No. 4,026,945 and U.S. Pat. No. 4,027,020, the entire contents of which are hereby incorporated by reference; mention may be made, for example, of polymers comprising the following repeating units:
   in which the radicals R¹, R², R³ and R⁴ which may be identical or different, denote a C₁-C₄ alkyl or hydroxyalkyl radical, n and p are integers ranging from 2 to 20 and X⁻ is an anion derived from a mineral or organic acid;
(11) Poly(quaternary ammonium) polymers consisting of repeating units of formula:
   in which p denotes an integer ranging from 1 to 6 approximately, D may be nothing or may represent a group —(CH₂)ᵣ—CO— in which r denotes a number equal to 4 or to 7, and X⁻ is an anion. Such polymers may be prepared according to the processes described in U.S. Pat. No. 4,157,388, U.S. Pat. No. 4,702,906, U.S. Pat. No. 4,719,282 and EP 122 324;
(12) Quaternary polymers of vinylpyrrolidone and of vinylimidazole;
(13) Polyamines of the polyethylene glycol (15) tallow polyamine type (CTFA dictionary name);
(14) Crosslinked methacryloyloxy (C₁-C₄) alkyltri (C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide. A crosslinked acrylamide/methacryloyloxyethyltrimethylammonium chloride copolymer (20/80 by weight) in the form of a dispersion containing 50% by weight of the said copolymer in mineral oil (Salcare® SC 92 from Ciba) can be used more particularly. A crosslinked methacryloyloxyethyltrimethylammonium chloride homopolymer containing about 50% by weight of the homopolymer in mineral oil or in a liquid ester (Salcare® SC 95, SC 96 from Ciba) can also be used.

Other cationic conditioning may include polyalkyleneimines, in particular polyethyleneimines, polymers containing vinylpyridine or vinylpyridinium units, condensates of polyamines and of epichlorohydrin, quaternary polyureylenes and chitin derivatives.

The amphoteric conditioning polymers (s) may be chosen especially from: polymers comprising units K and M randomly distributed in the polymer chain, in which K denotes a unit derived from a monomer comprising at least one basic nitrogen atom and M denotes a unit derived from an acidic monomer comprising one or more carboxylic or sulphonic groups; alternatively K and M may denote groups derived from zwitterionic carboxybetaine or sulphobetaine monomers; alternatively, K and M denote a cationic polymer chain comprising primary, secondary, tertiary or quaternary amine groups, in which at least one of the amine groups bears a carboxylic or sulphonic group linked via a hydrocarbon-based radical; alternatively K and M form part of a chain of a polymer containing an α,β-dicarboxylic ethylene unit in which one of the carboxylic groups has reacted with a polyamine comprising one or more primary or secondary amine groups.

The amphoteric conditioning polymers corresponding to the above definition that are more particularly preferred are chosen from the following polymers:
(1) polymers resulting from the copolymerization of a monomer derived from a vinyl compound bearing a carboxylic group such as, more particularly, (meth)acrylic acid, maleic acid, α-chloroacrylic acid, or else a dialkyldiallylammonium salt such as dimethyldiallylammonium chloride, and a basic monomer derived from a substituted vinyl compound containing at least one basic atom, such as, more particularly, dialkylaminoalkyl methacrylate and acrylate, dialkylaminoalkylmethacrylamide and -acrylamide, as described in U.S. Pat. No. 3,836,537. Mention may also be made of the sodium acrylate/acrylamidopropyltrimethylammonium chloride copolymer (Polyquart KE 3033 from Cognis) and the acrylic acid/dimethyldiallylammonium chloride copolymer (Merquat 280, 295, Plus 3330, from Nalco);
(2) polymers containing units derived from: a) at least one monomer chosen from (meth)acrylamides substituted on the nitrogen with an alkyl radical, in particular C₂-C₁₂, b) at least one acidic monomer containing one or more reactive carboxylic groups (for example (meth)acrylic acid, crotonic acid or itaconic acid, and monoesters of maleic or fumaric acids or anhydrides), and c) at least one basic monomer such as esters containing primary, secondary, tertiary and quaternary amine substituents of (meth)acrylic acid, fumaric acid or maleic acid, and the product of quaternization of dimethylaminoethyl methacrylate with dimethyl or diethyl sulphate. Octylacrylamide/acrylate/butylaminoethyl methacrylate copolymers (Amphomer or Lovocryl 47 by the company National Starch) are particularly used;
(3) Crosslinked and partially or totally alkylated polyaminoamides, derived from polyaminoamides of general formula —[CO—R⁵—CO-Z]- in which R⁵ is a divalent radical derived from a saturated or unsaturated dicarboxylic acid (for example adipic acid, 2,2,4-trimethyladipic acid, 2,4,4-trimethyladipic acid, terephthalic acid or itaconic acid), from an unsaturated monocarboxylic acid (for instance (meth) acrylic acid), from a C₁-C₆ alkyl ester of the abovementioned acids or from a radical derived from the addition of one of these acids to a bis-primary or bis-secondary amine, and Z denotes a radical of a bis-primary, mono- or bis-secondary polyalkylene-polyamine. Preferably, Z represents between 60 and 100 mol %, the radical —NH—[(CH₂)ₓ—NH]ₚ— with x=2 and p=2 or 3, or x=3 and p=2; this radical is derived from diethylenetriamine, from triethylenetetramine or from dipropylenetriamine; between 0 and 40 mol % the above radical, in which x=2 and p=1 and which is derived from ethylenediamine, or the radical derived from piperazine —N[CH₂CH₂]₂N—; between 0 and 20 mol %, the radical —NH—(CH₂)₆—NH— derived from hexamethylenediamine. The crosslinking agent for these polymers is a difunctional agent chosen from epihalohydrins, diepoxides, dianhydrides and bis-unsaturated derivatives, and alkylated by the action of acrylic acid, chloroacetic acid or an alkane sulfone or the alkali metal salts thereof;
(4) Polymers comprising at least zwitterionic units, for instance the butyl methacrylate/dimethylcarboxymethylammonioethyl methacrylate copolymer (Diaformer Z301 from Sandoz);
(5) Polymers derived from chitosan comprising monomer units corresponding to formulae (I), (II) and (III) below:
   with (I) representing from 0 to 30%, (II) from 5% to 50% and (III) from 30% to 90% in which R 6 represents a radical of formula:
   in which q denotes 0 or 1; and if q=0, R⁷, R⁸ and R⁹, which may be identical or different, represent a hydrogen, a methyl, hydroxyl, acetoxy, amino, monoalkylamino or dialkylamino group, optionally interrupted with one or more nitrogen atoms and/or optionally substituted with one or more amine, hydroxyl or carboxyl groups, alkylthio groups optionally bearing an amino group, or sulphonic group; or, if q=1, R⁷, R⁸ and R⁹, which may be identical or different, represent a hydrogen, and also the salts formed by these compounds with acids or bases;
(6) Polymers derived from the N-carboxyalkylation of chitosan, for instance N-carboxymethylchitosan or N-carboxybutylchitosan (Evalsan from Jan Dekker);
(7) Polymers as described in FR 1 400 366:
   in which R¹⁰ is a hydrogen, CH₃O—, CH₃CH₂O— or phenyl, R¹¹ and R¹⁴, which may be identical or different, represent a hydrogen or an alkyl radical (methyl or ethyl), R¹³ represents an alkyl radical (methyl or ethyl) or a radical of formula —R¹²—N(R¹⁴)₂, R¹² representing —(CH₂)₂—, —(CH₂)₃— or —CH₂—CH(CH₃)— and also the higher homologues of these radicals and containing up to 6 carbon atoms, and r is such that the molecular weight is between 500 and 6,000,000 and preferably between 1000 and 1,000,000;
(8) Amphoteric polymers of the type -D¹-X-D¹-X— chosen from:
   a) polymers obtained by the action of chloroacetic acid or sodium chloroacetate on compounds comprising at least one unit of formula -D¹-X-D¹-X-D¹- in which D¹ denotes a piperazinyl radical and X denotes the symbol E or E′, E or E′, which may be identical or different, denote a divalent radical which is an alkylene radical containing a straight or branched chain containing up to 7 carbon atoms in the main chain, optionally substituted with hydroxyl groups and possibly also comprising oxygen, nitrogen or sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups;
   (b) polymers of formula -D¹-X-D¹-X— in which D¹ denotes a piperazinyl radical and X denotes the symbol E or E′ and at least once E′; E having the meaning given above and E′ being a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily containing one or more carboxyl functions or one or more hydroxyl functions and betainized by reaction with chloroacetic acid or sodium chloroacetate;
(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also contain other vinyl comonomers such as vinylcaprolactam. Among the cationic or amphoteric conditioning polymers that may be used, the following are especially preferred:
   (a) among the cationic polymers:
      the dimethyldiallylammonium chloride homopolymer (Merquat 100 from Nalco);
      copolymers of dimethyldiallylammonium chloride and of acrylamide (Merquat 2200 from Nalco);
      polymers of poly(quaternary ammonium) type prepared and described in FR 2 270 846, consisting of repeating units of formulae (W) and (U) below:
      and especially those with a molecular weight, determined by gel permeation chromatography, of between 9500 and 9900;
      and especially those with a molecular weight, determined by gel permeation chromatography, of about 1200;
      polymers of poly(quaternary ammonium) type of the family (11) with X⁻ denoting chlorine, and especially those with a weight-average molecular mass of less than 100,000 and preferably less than or equal to 50,000;
   (b) among the amphoteric polymers:
      dimethyldiallylammonium chloride/acrylic acid copolymer (80/20) (Merquat 280 from Nalco-CTFA name: Polyquaternium 22);
      dimethyldiallylammonium chloride/acrylic acid copolymer (95/5) (Merquat 295 from Nalco);
      methacrylamidopropyltrimonium chloride, acrylic acid and ethyl acrylate copolymer (Merquat 2001 from Nalco-CTFA name: Polyquaternium 47);
      acrylamide/dimethyldiallylammonium chloride/acrylic acid terpolymer (Merquat Plus 3330 from Nalco-CTFA name: Polyquaternium 39).

Water-Soluble Styling Polymers

The water-soluble styling polymers useful herein includes; for example, vinylpyrrolidone homopolymers such as polyvinylpyrrolidone; vinylpyrrolidone copolymers such as polyvinylpyrrolidone/vinyl acetate copolymer and polyvinylpyrrolidone/dimethylaminoethylmethacrylate copolymer; dimethylaminoethylmethacrylate copolymer; polyquaterniums such as polyquaternium-11 and polyquaternium-4; alkyl esters of PVM/MA Copolymer such as ethyl ester of PVM/MA Copolymer and butyl ester of PVM/MA Copolymer; quaternized celluloses; acrylate homopolymers and acrylate copolymers such as carbomers; VA/crotonates/vinyl neodecanoate; poly(vinyl alcohol)-co-poly(vinyl acetate); and mixtures thereof. Some water-soluble styling polymers described herein can also be used as thickening agents.

The vinylpyrrolidone copolymers useful herein are those which comprise monomers other than vinylpyrrolidone. Non-limiting examples of vinylpyrrolidone copolymers useful herein include polyvinylpyrrolidone/acrylates/lauryl methacrylate copolymer, polyvinylpyrrolidone/dimethiconylacrylate/polycarbamyl/polyglycol ester, polyvinylpyrrolidone/dimethylaminoethylmethacrylate copolymer, polyvinylpyrrolidone/dimethylaminoethylmethacrylate/polycarbamylpolyglycol ester, polyvinylpyrrolidone/DMAPA acrylates copolymer, polyvinylpyrrolidone/eicosene copolymer, polyvinylpyrrolidone/hexadecene copolymer, polyvinylpyrrolidone/polycarbamyl polyglycol ester, polyvinylpyrrolidone/vinyl acetate copolymer, polyvinylpyrrolidone/vinyl acetate/itaconic acid copolymer, polyvinylpyrrolidone/vinyl acetate/vinyl propionate copolymer, and polyvinylpyrrolidone/vinyl caprolactam/DMAPA acrylates copolymer.

Preferably, the copolymer of pyrrolidone useful herein is a nonionic copolymer in view of less deteriorating conditioning benefits and better compatibility with thickening agent, especially carboxylic acid/carboxylate copolymer thickening agent.

Preferably, polyvinylpyrrolidone/vinyl acetate copolymer and polyvinylpyrrolidone/vinyl acetate/vinyl propionate copolymer are used in the compositions of the present invention, and more preferably, polyvinylpyrrolidone/vinyl acetate copolymer is used in the compositions of the present invention in view of less deteriorating conditioning benefits and better compatibility with thickening agent, especially carboxylic acid/carboxylate copolymer thickening agent. In the polyvinylpyrrolidone/vinyl acetate copolymer, the mole ratio of vinylpyrrolidone monomer to vinyl acetate monomer may be preferably from about 1:9 to about 9:1, more preferably from about 5:5 to about 8:2.

Commercially available vinylpyrrolidone copolymers useful herein include: CTFA name polyvinylpyrrolidone/vinyl acetate copolymer having tradenames Luviskol VA28E, Luviskol VA37E, Luviskol VA55E, Luviskol VA64E, Luviskol VA73E, Luviskol VA37HM, Luviskol VA64 Powder, Luviskol VA64W, and Luviskol VA73W, all available from BASF, and PVP/VA E series, I series, S-630, all available from ISP; CTFA name polyvinylpyrrolidone/vinyl acetate/vinyl propionate copolymer having tradename Luviskol VAP343E available from BASF; CTFA name polyvinylpyrrolidone/acrylates/lauryl methacrylate copolymer having tradename Acrylidone LM available from ISP; CTFA name polyvinylpyrrolidone/dimethiconylacrylate/polycarbamyl/polyglycol ester having tradename Pecogel S-1120 available from Phoenix; CTFA name polyvinylpyrrolidone/dimethylaminoethylmethacrylate copolymer having tradename Copolymer 845, Copolymer 937, and Copolymer 958, all available from ISP; CTFA name polyvinylpyrrolidone/dimethylaminoethylmethacrylate/polycarbamylpolyglycol ester having tradename Pecogel GC-310 and Pecogel GC-1110 available from Phoenix; CTFA name polyvinylpyrrolidone/DMAPA acrylates copolymer having tradename ACP-1163 available from ISP; CTFA name polyvinylpyrrolidone/eicosene copolymer having tradename Antaron V-220 and Ganex V-220 available from ISP; CTFA name polyvinylpyrrolidone/hexadecene copolymer having tradename Antaron V-216 and Ganex V-216 available from ISP; CTFA name polyvinylpyrrolidone/polycarbamyl polyglycol ester having tradename Pecogel A-12 and Pecogel H series available from Phoenix; CTFA name polyvinylpyrrolidone/vinyl caprolactam/DMAPA acrylates copolymer having tradename ACP-1189 available from ISP.

Thickening Agents

The thickening agents may include sodium alginate, gum arabic or cellulose derivatives such as methylcellulose, hydroxymethylcellulose, hydroxylethylcellulose, hydroxypropylcellulose or hydroxylpropylmethylcellulose, fatty alcohols, oxyethylenated or otherwise, cross linked polyacrylic acids.

The thickening agent will also include water soluble or water miscible polymers, have the ability to increase the viscosity of the composition, and are compatible with other components of the present invention.

Useful herein are carboxylic acid/carboxylate copolymers such as hydrophobically-modified cross-linked coplymers of carboxylic acid and alkyl carboxylate, and have an amphiphilic property. These carboxylic acid/carboxylate copolymers are obtained by copolymerizing 1) a carboxylic acid monomer such as acrylic acid, methacrylic acid, maleic acid, maleic anhydride, itaconic acid, fumaric acid, crotonic acid, or α-chloroacrylic acid, 2) a carboxylic ester having an alkyl chain of from 1 to about 30 carbons, and preferably 3) a crosslinking agent of the following formula: 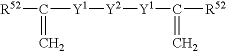
wherein R⁵² is a hydrogen or an alkyl group having from about 1 to about 30 carbons; Y¹, indepedently, is oxygen, CH₂O, COO, 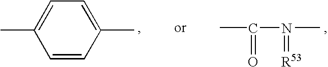
wherein R⁵³ is a hydrogen or an alkyl group having from about 1 to about 30 carbons; and y² is selected from (CH₂)m″ (CH₂CH₂O)m″, or (CH₂CH₂CH₂O)m″ wherein m″ is an integer of from 1 to about 30.

Suitable carboxylic acid/carboxylate copolymers herein are acrylic acid/alkyl acrylate copolymers having the following formula: 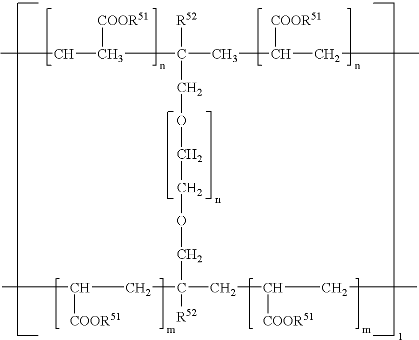
wherein R⁵¹, independently, is a hydrogen or an alkyl of 1 to 30 carbons wherein at least one of R⁵¹ is a hydrogen, R⁵² is as defined above, n, n′, m and m′ are integers in which n+n′+m+m′ is from about 40 to about 100, n″ is an integer of from 1 to about 30, and 1 is defined so that the copolymer has a molecular weight of about 500,000 to about 3,000,000.

Commercially available carboxylic acid/carboxylate copolymers useful herein include: CTFA name Acrylates/C10-30 Alkyl Acrylate Crosspolymer having tradenames Pemulene TR-1, Pemulene TR-2, Carbopol 1342, Carbopol 1382, and Carbopol ETD 2020, all available from B. F. Goodrich Company.

Neutralizing agents may be included to neutralize the carboxylic acid/carboxylate copolymers herein. Non-limiting examples of such neutralizing agents include sodium hydroxide, potssium hydroxide, ammonium hydroxide, monethanolamine, diethanolamine, triethanolamine, diisopropanolamine, aminomethylpropanol, tromethamine, tetrahydroxypropyl ethylenediamine, and mixtures thereof.

Another thickening agents useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Polyethylene glycols having a molecular weight of more than about 1000 are useful herein. Useful are those having the following general formula: 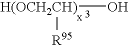
wherein R⁹⁵ is selected from the group consisting of H, methyl, and mixtures thereof. In the above structure, x³ has an average value of from about 1500 to about 25,000, preferably from about 2500 to about 20,000, and more preferably from about 3500 to about 15,000. When R⁹⁵ is H, these materials are polymers of ethylene oxide, which are also known as polyethylene oxides, polyoxyethylenes, and polyethylene glycols. Other useful polymers include the mixed polyethylene-polypropylene glycols, or polyoxyethylene-polyoxypropylene copolymer polymers. Polyethylene glycol polymers useful herein are PEG-2M wherein R⁹⁵ equals H and x³ has an average value of about 2,000 (PEG-2M is also known as Polyox WSR® N-10, which is available from Union Carbide and as PEG-2,000); PEG-5M wherein R⁹⁵ equals H and x³ has an average value of about 5,000 (PEG-5M is also known as Polyox WSR® N-35 and Polyox WSR® N-80, both available from Union Carbide and as PEG-5,000 and Polyethylene Glycol 300,000); PEG-7M wherein R⁹⁵ equals H and X³ has an average value of about 7,000 (PEG-7M is also known as Polyox WSR® N-750 available from Union Carbide); PEG-9M wherein R⁹⁵ equals H and x³ has an average value of about 9,000 (PEG 9-M is also known as Polyox WSR® N-3333 available from Union Carbide); and PEG-14 M wherein R⁹⁵ equals H and x³ has an average value of about 14,000 (PEG-14M is also known as Polyox WSR® N-3000 available from Union Carbide).

Other commercially available thickening agents highly useful herein include Carbomers with tradenames Carbopol 934, Carbopol 940, Carbopol 950, Carbopol 980, and Carbopol 981, all available from B. F. Goodrich Company, acrylates/steareth-20 methacrylate copolymer with tradename ACRYSOL 22 available from Rohm and Hass, nonoxynyl hydroxyethylcellulose with tradename AMERCELL POLYMER HM-1500 available from Amerchol, methylcellulose with tradename BENECEL, hydroxypropyl cellulose with tradename KLUCEL, all supplied by Herculus, hydroxyethyl cellulose with tradename NATROSOL 250HBR and 250 MBR available from Aqualon, ethylene oxide and/or propylene oxide based polymers with tradenames CARBOWAX PEGs, POLYOX WASRs, and UCON FLUIDS, all supplied by Amerchol.

Other thickening agents useful herein are hydrophobically modified cellulose ethers. The hydrophobically modified cellulose ethers useful herein are preferably nonionic polymers. The hydrophobically modified cellulose ethers useful herein comprise a hydrophilic cellulose backbone and a hydrophobic substitution group. The hydrophilic cellulose backbone has a sufficient degree of nonionic substitution to cellulose to be water soluble. Such hydrophilic cellulose backbone is selected from the group consisting of methyl cellulose, hydroxymethyl cellulose, hydroxyethyl cellulose, hydroxyethyl ethylcellulose, hydroxypropyl cellulose, hydroxypropyl methylcellulose, and mixtures thereof. The amount of nonionic substitution is not critical, so long as there is an amount sufficient to assure that the hydrophilic cellulose backbone is water soluble. The hydrophilic cellulose backbone has a molecular weight of about less than 800,000, preferably from about 20,000 to about 700,000, or from about 75 D. P. to about 2500 D. P. Further, where a high viscosity building effect is not desirable, a lower molecular weight cellulose backbone is preferred. One of the preferred hydrophilic cellulose backbone is hydroxyethyl cellulose having a molecular weight of from about 50,000 to about 700,000. Hydroxyethyl cellulose of this molecular weight is known to be one of the most hydrophilic of the materials contemplated. Thus, hydroxyethyl cellulose can be modified to a greater extent than other hydrophilic cellulose backbones.

The hydrophilic cellulose backbone is further substituted with a hydrophobic substitution group via an ether linkage to render the hydrophobically modified cellulose ether to have less than 1% water solubility, preferably less than 0.2% water solubility. The hydrophobic substitution group is selected from a straight or branched chain alkyl group of from about 10 to about 22 carbons; wherein the ratio of the hydrophilic groups in the hydrophilic cellulose backbone to the hydrophobic substitution group being from about 2:1 to about 1000:1, preferably from about 10:1 to about 100:1.

Commercially available hydrophobically modified cellulose ethers useful herein include: cetyl hydroxyethylcellulose having tradenames NATROSOL PLUS 330CS and POLYSURF 67, both available from Aqualon Company, Del., USA, having cetyl group substitution of about 0.4% to about 0.65% by weight of the entire polymer.

Polyorganosiloxanes

The polyorganosiloxanes may include cyclic volatile silicones and non-volatile silicones selected from the group consisting of:
(i) polyalkylsiloxanes;
(ii) polyarylsiloxanes;
(iii) polyalkylarylsiloxanes;
(iv) silicone gums;
(v) silicone resins;
(vi) organomodified polyorganosiloxanes containing, in their general structure, one or more organofunctional groups directly attached to the siloxane chain or attached via a hydrocarbon-based radical;
(vii) block copolymers having a polysiloxanepolyalkylene linear block as repeating unit;
(viii) grafted silicone polymers containing a non-silicone organic skeleton, consisting of an organic main chain formed from organic monomers not containing silicone, onto which is grafted, within the said chain as well as, optionally, on at least one of its ends, at least one polysiloxane macromonomer;
(ix) grafted silicone polymers containing a polysiloxane skeleton, grafted with non-silicone organic monomers, comprising a polysiloxane main chain onto which is grafted, within the said chain as well as, optionally, on at least one of its ends, at least one organic macromonomer containing no silicone;
(x) and mixtures thereof.

Inorganic Materials

Suitable inorganic materials which may be used as cosmetic additives include, but are not limited to, inorganic salts, pigments and coloring agents, inorganic spherical particles, hollow spherical particles, minerals, fillers, clay-type materials, and mixtures thereof.

Inorganic Salts

The inorganic salts may include phosphates, sulfates, nitrates, citrates, halides, gluconates, fumarates, oxalates, and oxides. The counter ions of such salts can be, but are not limited to, sodium, potassium, ammonium, magnesium, aluminum, calcium, zinc, selenium, iron, copper and manganese or other mono-, di- and tri-valent cations.

Pigments and Coloring Agents

The inorganic pigments and coloring agents of the compositions may include iron oxides, ultramarine and chromium or chromium hydroxide colors, zirconium oxide, cerium oxide, manganese violet, ultramarine blue, chromium hydrate, ferric blue, chromium oxide, and metallic pigments such as aluminum and bronze, pearlescent materials, and mixtures thereof. Inorganic pigments may also include titanium dioxide, which has optionally been surface-treated.

Other pigments and coloring agents include magnesium carbonate, calcium carbonate, magnesium silicate, aluminum magnesium silicate, silica, zinc oxide, red iron oxide, brown iron oxide, yellow iron oxide, black iron oxide, ferric ammonium ferrocyanide, titanated mica, iron oxide titanated mica, bismuth oxychloride.

Inorganic Spherical Particles

Inorganic spherical particles may include spherical silica particles available in various particle sizes and porosities including MSS-500/H, MSS-500/3H, MSS-500, MSS-500/3, MSS-500/N and MSS-500/3N available from KOBO Products Inc.; those available from Presperse Inc. under the trade name Spheron including Spheron N-2000, P-1000, P-1500 and L-1500, and those available from Sunjin Chemical Co. under the trade name Sunsil including Sunsil 20, 20L, 20H, 50L, 50, 50H, 130L, 130 and 130H. Other non-limiting examples of spherical inorganic particles useful in the present invention include various silicates including magnesium silicate such as those available from 3M under the trade name CM-111 Cosmetic Microspheres, and glass spheres such as those available from Nippon Paint Corp. under the trade name PrizmaLite Glass Spheres and hollow silica microspheres (Silica Beads® from Maprecos). Some types of inorganic spherical particles may also be considered as fillers.

Hollow Spherical Particles

The hollow spherical particles include those whose walls are formed from an inorganic material. The inorganic material may be a silica, a soda-lime-borosilicate glass, a silica-alumina ceramic, or an alkali alumino silicate ceramic. Non-limiting examples of commercially available suitable low density, inorganic particles are H50/10,000 EPX (particle size range approximately 20-60 μm), S38 (particle size range approximately 15-65 μm), W-210 (particle size range approximately 1-12 μm), W410 (particle size range approximately 1-24 μm), W-610 (particle size range approximately 1-40 μm), G-200 (particle size range approximately 1-12 μm), G400 (particle size range approximately 1-24 μm), G-600 (particle size range approximately 140 μm), all of which are marketed under the trademarks 3M™ Scotchlite® Glass Bubbles, 3M® Zeeospheres® ceramic microspheres, and 3M® Z-Light Spheres® Ceramic Microspheres. Also useful are hollow calcium aluminum borosilicate spheres and calcium sodium borosilicate marketed as LUXSIL® (3-13 μm mean diameter) available from PQ Corporation. Some types of hollow spherical particles may also be considered as fillers.

Minerals

Minerals may include calcium carbonate, calcium oxalate, and calcium polyphosphates.

Fillers

The inorganic fillers may be of any shape, in platelet form, lamellar structures, spherical or oblong, irrespective of the crystallographic shape (for example leaflet, cubic, hexagonal, orthorhombic, etc.). Inorganic lamellar fillers are chosen from talcs, hydrated magnesium silicates, micas such as titanium micas, aluminosilicates, clays such as sericites, kaolin, hydrated aluminum silicate and boron nitrides. Inorganic spherical fillers are chosen from zinc oxides, titanium oxides, precipitated calcium carbonate, magnesium carbonate and hydrocarbonate, non-porous spherical silica, hydroxyapatite, silica microspheres with open or hollow porosity, optionally impregnated with a cosmetic active agent and glass and ceramic microcapsules.

Other fillers include precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms, preferably from 12 to 18 carbon atoms, for example zinc stearate, magnesium stearate, lithium stearate, zinc laurate or magnesium myristate.

Clay-Type Materials

Inorganic Clay-type materials may include bentonite, ceramic, kaolin, slip clays, polyorganosilicates, kaolinite, montmorillonite, atapulgite, illite, bentonite, halloysite, silica, alumina, mudds.

The cosmetic additive will typically be present in the composition in an amount of from 0.05% to 20%, preferably from about 0.1% to about 10%, more preferably from about 1% to about 5% based on the weight of the composition.

In the event a cosmetic additive is employed, the resulting nanocomposite, when exposed to a flash of light, will result in the formation of a thin film on the keratinous substrate. The flash-welded film may later be easily removed from the substrate with water. The invention may also apply to other keratinous and biological surfaces such as human nails and skin. Desirable aesthetic visual and cosmetic properties such as gloss or shine and coloration effects can be imparted to solid substrates such as human nails.

Adjuvants

The cosmetic composition may also comprise an effective amount of other agents, which are previously known elsewhere in the treatment of human keratin fibers, such as various common adjuvants, for instance surfactants that are well known in the prior art and of anionic, cationic, nonionic, amphoteric or zwitterionic type or mixtures thereof, antioxidants, fragrances, dispersants, opacifiers, sequestering agents such as EDTA and etidronic acid, organic UV-screening agents, waxes, volatile or nonvolatile, cyclic or linear or branched silicones, which are organomodified (especially with amine groups) or unmodified, preserving agents, ceramides, pseudoceramides, plant, mineral or synthetic oils, and vitamins or provitamins, for instance panthenol.

Surfactants

Preferably, the cosmetic composition according to the invention comprises one or more surfactants that may be chosen, without preference, alone or as mixtures, from anionic, amphoteric, nonionic, zwitterionic and cationic surfactants.

The surfactants that are suitable for carrying out the present invention are especially the following:

(i) Anionic Surfactant(s):
As examples of anionic surfactants which can be used, alone or as mixtures, in the context of the present invention, mention may be made in particular (nonlimiting list) of salts (in particular alkaline salts, especially sodium salts, ammonium salts, amine salts, amino alcohol salts or magnesium salts) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; (C₆-C₂₄) alkyl sulfosuccinates, (C₆-C₂₄) alkyl ether sulfosuccinates, (C₆-C₂₄) alkylamide sulfosuccinates; (C₆-C₂₄) alkyl sulfoacetates; (C₆-C₂₄) acyl sarcosinates and (C₆-C₂₄) acyl glutamates. It is also possible to use the carboxylic esters of (C₆-C₂₄) alkylpolyglycosides, such as alkylglucoside citrates, alkylpolyglycoside tartrates and alkylpolyglycoside sulfosuccinates, alkylsulfosuccinamates; acyl isethionates and N-acyltaurates, the alkyl or acyl radical of all of these various compounds preferably containing from 12 to 20 carbon atoms, and the aryl radical preferably denoting a phenyl or benzyl group. Among the anionic surfactants which can also be used, mention may also be made of fatty acid salts such as the salts of oleic, ricinoleic, palmitic and stearic acids, coconut oil acid or hydrogenated coconut oil acid; acyl lactylates in which the acyl radical contains 8 to 20 carbon atoms. Alkyl-D-galactosideuronic acids and their salts, polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, in particular those containing from 2 to 50 alkylene oxide, in particular ethylene oxide, groups, and mixtures thereof can also be used.

(ii) Nonionic Surfactant(s):
The nonionic surfactants are also compounds that are well known per se (see in particular in this respect “Handbook of Surfactants” by M. R. Porter, published by Blackie & Son (Glasgow and London), 1991, pp. 116-178) and, in the context of the present invention, their nature is not a critical feature. Thus, they can be chosen in particular from (nonlimiting list) polyethoxylated or polypropoxylated alkylphenols, α-diols or alcohols having a fatty chain containing, for example, 8 to 18 carbon atoms, it being possible for the number of ethylene oxide or propylene oxide groups to range in particular from 2 to 50. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; polyethoxylated fatty amines preferably having from 2 to 30 mol of ethylene oxide; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides.

(iii) Amphoteric or Zwitterionic Surfactant(s):
The amphoteric or zwitterionic surfactants, whose nature is not a critical feature in the context of the present invention, can be, in particular (nonlimiting list), aliphatic secondary or tertiary amine derivatives in which the aliphatic radical is a linear or branched chain containing 8 to 18 carbon atoms and containing at least one water-soluble anionic group (for example carboxylate, sulfonate, sulfate, phosphate or phosphonate); mention may also be made of (C₈-C₂₀) alkylbetaines, sulfobetaines, (C₈-C₂₀) alkylamido (C₁-C₆) alkylbetaines or (C₈-C₂₀) alkylamido (C₁-C₆) alkylsulfobetaines.

Among the amine derivatives, mention may be made of the products sold under the name Miranol, as described in U.S. Pat. Nos. 2,528,378 and 2,781,354, the entire contents of which are hereby incorporated by reference, and classified in the CTFA dictionary, 3rd edition, 1982, under the names amphocarboxyglycinates and amphocarboxypropionates of respective structures:
R₂—CONHCH₂CH₂—N(R₃)(R₄)(CH₂COO⁻)
in which: R₂ denotes an alkyl radical of an acid R₂—COOH present in hydrolyzed coconut oil, a heptyl, nonyl or undecyl radical, R₃ denotes a β-hydroxyethyl group and R₄ denotes a carboxymethyl group; and
R₂′—CONHCH₂CH₂—N(B)(C)
in which:
B represents —CH₂CH₂OX′, C represents —(CH₂)_{z}—Y′, with z=1 or 2,
X′ denotes a—CH₂CH₂—COOH group or a hydrogen atom,
Y′ denotes —COOH or a—CH₂—CHOH—SO₃H radical,
R₂′ denotes an alkyl radical of an acid RG-COOH present in coconut oil or in hydrolyzed linseed oil, an alkyl radical, in particular a C₇, C₉, C₁₁ or C₁₃ alkyl radical, a C₁₇ alkyl radical and its iso form, an unsaturated C₁₇ radical.

These compounds are classified in the CTFA dictionary, 5th edition, 1993, under the names disodium coco-ampho-diacetate, disodium lauro-ampho-diacetate, disodium capryl-ampho-diacetate, disodium caprylo-ampho-diacetate, disodium coco-ampho-dipropionate, disodium lauro-ampho-dipropionate, disodium capryl-ampho-dipropionate, disodium caprylo-ampho-dipropionate, lauro-ampho-dipropionic acid and coco-ampho-dipropionic acid.

By way of example, mention may be made of the cocoamphodiacetate sold under the trade name Miranol® C2M Concentrate by the company Rhodia Chimie.

(iv) Cationic Surfactants:
Among the cationic surfactants that may be mentioned in particular (nonlimiting list) are: primary, secondary or tertiary fatty amine salts, optionally polyoxyalkylenated; quaternary ammonium salts such as tetraalkylammonium, alkylamidoalkyl-trialkylammonium, trialkylbenzylammonium, trialkylhydroxyalkylammonium or alkylpyridinium chlorides or bromides; imidazoline derivatives; or amine oxides of cationic nature.

The amount of surfactants present in the composition according to the invention may range from 0.01 to 40% by weight and preferably from 0.5 to 30% by weight, relative to the total weight of the composition.

Flash-Welding

According to the process of the present invention, a visual and cosmetic effect may be imparted onto a keratinous substrate by contacting the substrate with the above-disclosed composition and then flash-welding the composition onto the surface of the substrate.

Flash-welding is conducted by irradiating the keratinous substrate treated with the cosmetic composition with a sudden flash of light. Examples of suitable flashes of light which may be employed include, but are not limited to, those emanating from a camera flash or a flashlight employed within a few centimeters from the keratinous substrate. The heat from the camera flash or flashlight welds or melts the conductive polymer nanofiber onto the surface of the keratinous substrate or to another polymer or inorganic material applied separately to the substrate, to form a thin polymer film or nano-composite film on the surface thereof. The source of the high intensity light is not limited to a camera flash or flashlight. A more detailed description concerning the mechanisms associated with flash-welding is found in (Huang and Kaner, 2004, Nature Materials, 3, 783-786), the entire content of which is hereby incorporated by reference.

The invention will be better understood by the following examples, all of which are intended for illustrative purposes only, and are not meant to unduly limit the scope of the invention in any way.

### EXAMPLES

Part 1: Conductive Polymer Nanofibers

### Example 1

### Styling Mousse

| Ingredient | Percentage (%) |
|---|---|
| Poly(aniline) Nanofibers | 0.5% |
| Empicol BSD 52 (Sodium Laureth-8 Sulfate and | 0.3% |
| Sodium Laureth Sulfate and Magnesium Laureth-8 | |
| Sulfate and Magnesium Laureth Sulfate and Sodium | |
| Oleth Sulfate and Magnesium Oleth Sulfate) | |
| Water | q.s. to 100% |
| Propellant A46 (Isobutane and Propane) | 6.0% |

### Example 2

### Styling Mousse

| Ingredient | Percentage (%) |
|---|---|
| Poly(aniline) Nanofibers | 0.5% |
| Empicol BSD 52 (Sodium Laureth-8 Sulfate and | 0.2% |
| Sodium Laureth Sulfate and Magnesium Laureth-8 | |
| Sulfate and Magnesium Laureth Sulfate and Sodium | |
| Oleth Sulfate and Magnesium Oleth Sulfate) | |
| Water | q.s. to 100% |
| Propellant A46 (Isobutane and Propane) | 10.0% |

Part 2: Conductive Polymer Nanofibers with Film-Forming Polymers

### Example 3

### Styling Mousse

| Ingredient | Percentage (%) |
|---|---|
| Poly(aniline) Nanofibers | 0.5% |
| Poly(vinyl alcohol)-co-Poly(vinyl acetate) | 0.5% |
| Empicol BSD 52 (Sodium Laureth-8 Sulfate and | 0.3% |
| Sodium Laureth Sulfate and Magnesium Laureth-8 | |
| Sulfate and Magnesium Laureth Sulfate and Sodium | |
| Oleth Sulfate and Magnesium Oleth Sulfate) | |
| Water | q.s. to 100% |
| Propellant A46 (Isobutane and Propane) | 6.0% |

### Example 4

### Styling Mousse

| Ingredient | Percentage (%) |
|---|---|
| Poly(aniline) Nanofibers | 0.5% |
| Aquaflex FX-64 | 2.0% |
| (Isobutylene/Ethylmaleimide/Hydroxyethylmaleimide | |
| Copolymer | |
| Empicol BSD 52 (Sodium Laureth-8 Sulfate and | 0.3% |
| Sodium Laureth Sulfate and Magnesium Laureth-8 | |
| Sulfate and Magnesium Laureth Sulfate and Sodium | |
| Oleth Sulfate and Magnesium Oleth Sulfate) | |
| Water | q.s. to 100% |
| Propellant A46 (Isobutane and Propane) | 6.0% |

Part 3: Conductive Polymer Nanofibers with Adhesive Micron-Objects

### Example 5

### Styling Mousse

| Ingredient | Percentage (%) |
|---|---|
| Poly(aniline) Nanofibers | 0.5% |
| Gel-Tac ® 205D (2-Ethylhexyl Acrylate) | 2.0% |
| Empicol BSD 52 (Sodium Laureth-8 Sulfate and | 0.3% |
| Sodium Laureth Sulfate and Magnesium Laureth-8 | |
| Sulfate and Magnesium Laureth Sulfate and Sodium | |
| Oleth Sulfate and Magnesium Oleth Sulfate) | |
| Water | q.s. to 100% |
| Propellant A46 (Isobutane and Propane) | 6.0% |

Part 4: Conductive Polymer Nanofibers with Non-Adhesive Polymer Beads

### Example 6

### Styling Mousse

| Ingredient | Percentage (%) |
|---|---|
| Poly(aniline) Nanofibers | 0.5% |
| Poly(styrene)-core/Poly(methyl | 2.0% |
| methacrylate)-shell, cross-linked | |
| Empicol BSD 52 (Sodium Laureth-8 Sulfate and | 0.3% |
| Sodium Laureth Sulfate and Magnesium Laureth-8 | |
| Sulfate and Magnesium Laureth Sulfate and Sodium | |
| Oleth Sulfate and Magnesium Oleth Sulfate) | |
| Water | q.s. to 100% |
| Propellant A46 (Isobutane and Propane) | 6.0% |

Part 5: Conductive Polymer Nanofibers with Non-Adhesive Inorganic Particles

### Example 7

### Styling Mousse

| Ingredient | Percentage (%) |
|---|---|
| Poly(aniline) Nanofibers | 0.5% |
| Luxsil ® (Calcium Sodium Borosilicate) | 0.1% |
| Empicol BSD 52 (Sodium Laureth-8 Sulfate and | 0.2% |
| Sodium Laureth Sulfate and Magnesium Laureth-8 | |
| Sulfate and Magnesium Laureth Sulfate and Sodium | |
| Oleth Sulfate and Magnesium Oleth Sulfate) | |
| Water | q.s. to 100% |
| Propellant A46 (Isobutane and Propane) | 6.0% |

### Example 8

### Styling Mousse

| Ingredient | Percentage (%) |
|---|---|
| Poly(aniline) Nanofibers | 0.5% |
| Biron ® Liquid Silver (Bismuth Oxychloride) | 0.1% |
| Empicol BSD 52 (Sodium Laureth-8 Sulfate and | 0.2% |
| Sodium Laureth Sulfate and Magnesium Laureth-8 | |
| Sulfate and Magnesium Laureth Sulfate and Sodium | |
| Oleth Sulfate and Magnesium Oleth Sulfate) | |
| Water | q.s. to 100% |
| Propellant A46 (Isobutane and Propane) | 6.0% |

Part 6: Conductive Polymer Nanofibers with Coloring Agents

### Example 9

### Styling Mousse

| Ingredient | Percentage (%) |
|---|---|
| Poly(aniline) Nanofibers | 0.5% |
| Basazol C Violet PR 8055 | 0.5% |
| Empicol BSD 52 (Sodium Laureth-8 Sulfate and | 0.3% |
| Sodium Laureth Sulfate and Magnesium Laureth-8 | |
| Sulfate and Magnesium Laureth Sulfate and Sodium | |
| Oleth Sulfate and Magnesium Oleth Sulfate) | |
| Water | q.s. to 100% |
| Propellant A46 (Isobutane and Propane) | 6.0% |

1. A composition comprising:
(a) a cosmetically acceptable medium;
(b) at least one conductive polymer nanofiber present in an amount sufficient to impart a visual and cosmetic effect to a keratinous surface when irradiated with a flash of light; and
(c) optionally, at least one cosmetic additive for imparting an additional cosmetic effect onto the keratinous surface.

2. The composition of claim 1 wherein the conductive polymer nanofiber has a diameter of about 100 nm or less.

3. The composition of claim 1 wherein the conductive polymer nanofiber is present in an amount of from about 0.01% to about 50% by weight, based on the weight of the composition.

4. The composition of claim 1 wherein the conductive polymer nanofiber is present in an amount of from about 0.01% to about 2% by weight, based on the weight of the composition.

5. The composition of claim 1 wherein the conductive polymer nanofiber is present in an amount of from about 0.1% to about 1% by weight, based on the weight of the composition.

6. The composition of claim 1 wherein the conductive polymer nanofiber is a polyaniline nanofiber.

7. The composition of claim 1 wherein the cosmetic additive is a polymer.

8. The composition of claim 7 wherein the polymer is selected from fixative polymers, conditioning polymers, water-soluble polymers, polymer beads, thickening agents, polyorganosiloxanes, and mixtures thereof.

9. The composition of claim 7 wherein the polymer is present in an amount of from about 0.05% to about 20%, based on the weight of the composition.

10. The composition of claim 7 wherein the polymer is present in an amount of from about 0.1% to about 10%, based on the weight of the composition.

11. The composition of claim 7 wherein the polymer is present in an amount of from about 1% to about 5%, based on the weight of the composition.

12. The composition of claim 1 wherein the cosmetic additive is an inorganic material.

13. The composition of claim 12 wherein the inorganic material is selected from inorganic salts, pigments and coloring agents, inorganic spherical particles, hollow spherical particles, minerals, fillers, clay-type materials, and mixtures thereof.

14. The composition of claim 12 wherein the inorganic material is present in an amount of from about 0.05% to about 20%, based on the weight of the composition.

15. The composition of claim 12 wherein the inorganic material is present in an amount of from about 0.1% to about 10%, based on the weight of the composition.

16. The composition of claim 12 wherein the inorganic material is present in an amount of from about 1% to about 5%, based on the weight of the composition.

17. A composition comprising:
(a) from about 0.1% to about 1% by weight of a polyaniline nanofiber;
(b) a cosmetically acceptably medium; and
(c) at least one cosmetic additive chosen from a polymer and an inorganic material.

18. A process for imparting a visual and cosmetic effect onto a keratinous substrate comprising:
(a) providing a keratinous substrate;
(b) providing a cosmetic composition containing:
(i) a cosmetically acceptable medium;
(ii) at least one conductive polymer nanofiber; and
(iii) optionally, at least one cosmetic additive for imparting an additional cosmetic effect onto the keratinous substrate;
(c) contacting the keratinous substrate with the cosmetic composition to form a treated keratinous substrate; and
(d) irradiating the treated keratinous substrate with a flash of light, thereby flash-welding the composition onto the keratinous substrate.

19. The process of claim 18 wherein the conductive polymer nanofiber has a diameter of about 100 nm or less.

20. The process of claim 18 wherein the conductive polymer nanofiber is present in an amount of from about 0.01% to about 50% by weight, based on the weight of the composition.

21. The process of claim 18 wherein the conductive polymer nanofiber is present in an amount of from about 0.01% to about 2% by weight, based on the weight of the composition.

22. The process of claim 18 wherein the conductive polymer nanofiber is present in an amount of from about 0.1% to about 1% by weight, based on the weight of the composition.

23. The process of claim 18 wherein the conductive polymer nanofiber is a polyaniline nanofiber.

24. The process of claim 18 wherein the cosmetic additive is chosen from a polymer, an inorganic material, and mixtures thereof.

25. The process of claim 24 wherein the cosmetic additive is a polymer selected from fixative polymers, conditioning polymers, water-soluble polymers, polymer beads, thickening agents, polyorganosiloxanes, and mixtures thereof.

26. The process of claim 25 wherein the polymer is present in an amount of from about 0.05% to about 20%, based on the weight of the composition.

27. The process of claim 25 wherein the polymer is present in an amount of from about 0.1% to about 10%, based on the weight of the composition.

28. The process of claim 25 wherein the polymer is present in an amount of from about 1% to about 5%, based on the weight of the composition.

29. The process of claim 24 wherein the cosmetic additive is an inorganic material selected from inorganic salts, pigments and coloring agents, inorganic spherical particles, hollow spherical particles, minerals, fillers, clay-type materials, and mixtures thereof.

30. The process of claim 29 wherein the inorganic material is present in an amount of from about 0.05% to about 20%, based on the weight of the composition.

31. The process of claim 29 wherein the inorganic material is present in an amount of from about 0.1% to about 10%, based on the weight of the composition.

32. The process of claim 29 wherein the inorganic material is present in an amount of from about 1% to about 5%, based on the weight of the composition.

33. The process of claim 18 wherein the conductive polyaniline nanofibers is present in an amount of from about 0.1% to about 1% and the polymer is present in an amount of from about 1% to about 5%, based on the weight of the composition, in a cosmetically acceptable medium.

34. A process for imparting a visual and cosmetic effect onto a keratinous substrate comprising:
(a) providing a keratinous substrate;
(b) providing a cosmetic composition containing:
(i) a cosmetically acceptable medium;
(ii) from about 0.1% to about 1% by weight of at least one polyaniline nanofiber; and
(iii) from about 1% to about 5% by weight of at least one cosmetic additive for imparting an additional cosmetic effect onto the keratinous substrate, all weights based on the weight of the composition;
(c) contacting the keratinous substrate with the cosmetic composition to form a treated keratinous substrate; and
(d) irradiating the treated keratinous substrate with a flash of light, thereby flash-welding the composition onto the keratinous substrate.

35. A composition comprising:
(a) a cosmetically acceptable medium;
(b) at least one conductive polymer nanofiber present in an amount sufficient to impart visual and cosmetic effects to a human nail surface when irradiated with a flash of light; and
(c) optionally, at least one cosmetic additive for imparting an additional cosmetic effect onto the human nail surface.

36. A process for imparting visual and cosmetic effects onto a human nail surface comprising:
(a) providing a human nail surface;
(b) providing a cosmetic composition containing:
(i) a cosmetically acceptable medium;
(ii) from about 0.1% to about 50% by weight of at least one polyaniline nanofiber; and
(iii) from about 0.01% to about 5% by weight of at least one cosmetic additive for imparting an additional cosmetic effect onto the human nail surface, all weights based on the weight of the composition;
(c) contacting the human nail surface with the cosmetic composition to form a treated human nail surface; and
(d) irradiating the treated human nail surface with a flash of light, thereby flash-welding the composition onto the human nail surface.
